(19) United States
(12) Patent Application Publication
(10) Pub. No.: US 2024/0294860 A1
(43) Pub. Date: Sep. 5, 2024

(54) MACRO-SPARGER FOR BENCHTOP BIOREACTORS

(71) Applicant: CORNING INCORPORATED, CORNING, NY (US)

(72) Inventor: Nasim Hashemi, Groton, MA (US)

(21) Appl. No.: 18/573,196

(22) PCT Filed: Jun. 14, 2022
(86) PCT No.: PCT/US2022/033353

§ 371 Date: Dec. 21, 2023

Related U.S. Application Data
(60) Provisional application No. 63/216,761, filed on Jun. 30, 2021.

(51) Int. Cl. C12M 1/00

(52) U.S. Cl. CPC C12M 29/06; C12M 29/10

(57) ABSTRACT

A macrosparger (300) for a benchtop bioreactor (370) comprises a tubing (310) and a base (330). The base comprises a plurality of sparging elements (320) in gaseous communication with the tubing and an interior of a benchtop bioreactor. Each sparging element comprises a cylindrical body having an internal compartment formed by a top surface and a bottom surface, the top surface and bottom surface connected by a sidewall, wherein the top surface and bottom surface are substantially oval-shaped. The benchtop bioreactor may be a perfusion bioreactor. A related system comprising the macrosparger and benchtop bioreactor may be used for performing a continuous cell culture.

### CROSS-REFERENCE TO RELATED APPLICATIONS

This application claims the benefit of priority under 35 U.S.C. § 119 of U.S. Provisional Application Ser. No. 63/216,761 filed on Jun. 30, 2021, the content of which is relied upon and incorporated herein by reference in its entirety.

### TECHNICAL FIELD

The present disclosure generally relates to the bioprocess field and particularly relates to a macrosparger for use in benchtop bioreactors.

### BACKGROUND

Bioprocess procedures include the upstream and downstream processes associated with the production of therapeutic products of interest from cultured cells, which may be mammalian, insect, or microbial cells. Bioprocess procedures in the biopharmaceutical industry are changing in order to reduce the cost of goods, increase the speed of production, and improve flexibility in production. One example of a biopharma industry change is the use of next generation approaches, such as using perfusion-based benchtop bioreactors for seed train intensification. Some intensified cell culture processes produce biopharmaceuticals in benchtop scale bioreactors with high volumetric production rates. However, such technologies are difficult to design, especially when production may require the use of mammalian cells that are sensitive to shear.

In addition, as cell densities continue to increase within benchtop bioreactors, the need for higher mass transfer rate and more efficient mixing will increase. Therefore, benchtop stirred bioreactors may provide more homogenous flow and higher mass transfer rate for applications like seed train intensification. In such a system, gas-liquid mass transfer may be achieved through super-surface (headspace) methods or sub-surface (sparging) methods. In some applications, aeration through headspace is enough to meet the gas-liquid mass transfer demand. However, computational fluid dynamics (CFD) results and published data indicate that oxygen transfer from headspace is insufficient for meaningful cell density.

In a benchtop stirred bioreactor, oxygen transfer may be affected by many variables, particularly sparger configuration and superficial gas velocity. Mammalian cells that are shear sensitive require a gentle aeration. Due to the need for gentle aeration, spargers for intensified processes must factor geometry, hole diameter, hole counts, and gas entrance velocity (GEV) into the design. Selection criteria of sparger types may also vary based on desired performance and customer needs. Commercially available benchtop bioreactors are equipped with different aeration configurations including pipe sparger, drilled hole spargers, and microspargers.

For seed train intensification uses, commercially available benchtop bioreactors typically rely on microspargers, which create micro-sized bubbles (e.g., having a diameter less than or equal to about 0.5 mm) efficient at delivering oxygen. However, microspargers suffer from many drawbacks. For example, CFD results for a 500 mL spinner flask indicated that a bioreactor facilitated with a microsparger exhibited lower mass transfer rate when compared with a bioreactor facilitated with a drilled hole sparger. The CFD results are due to the presence of a high number of bubbles created by the microsparger, which facilitates the cohesion of the bubbles in the small working volume of the benchtop bioreactor and negatively affects mass transfer rate. Another issue involves difficulty in controlling the gas entrance velocity emitted from the microsparger, which may be a significant cause of cell death in bioreactors. In addition, microspargers suffer from an excessive formation of foam above the liquid surface, which results in reduced productivity in benchtop bioreactors.

Furthermore, commercially available macrospargers for use in benchtop bioreactors typically consist of a length of pipe or tubing with one or multiple openings which direct the flow of gas into the bioreactor. The diameters of the openings are on the order of millimeters, which result in formation of larger bubbles than microsparger options. However, the increased bubble size decreases the gas-liquid interfacial surface area, which leads to decreased mass transfer rate, which is undesirable in high cell density culture. Therefore, a need exists for an aeration device for a benchtop bioreactor that provides a high mass transfer rate with minimal foam and shear.

### SUMMARY

In an aspect, embodiments of the disclosure are directed to a macrosparger for use in benchtop bioreactors. Macrospargers according to embodiments described herein allow for a higher mass transfer rate with less shear damage and foam formation compared to commercially available spargers typically used in benchtop bioreactors. Macrospargers according to embodiments of the disclosure comprise a plurality of sparging elements for aeration that are independently controllable. For example, the gas flow rate, number of holes, and size of holes are independently controllable for each sparging element.

In an aspect, embodiments of the disclosure are directed to a macrosparger for a benchtop bioreactor comprising a tubing and a base. The base comprises a plurality of sparging elements in gaseous communication with the tubing and an interior of a benchtop bioreactor. Each sparging element comprises a cylindrical body having an internal compartment formed by a top surface and a bottom surface, the top surface and bottom surface connected by a sidewall, wherein the top surface and bottom surface are substantially oval-shaped.

In some embodiments, the top surface and bottom surface are substantially flat. In some embodiments, the top surface comprises a plurality of openings, the compartment in gaseous communication with the interior of the benchtop bioreactor through the plurality of openings. In some embodiments, each of the openings in the plurality of openings is uniformly spaced or uniformly disposed on the top surface.

In some embodiments, the top surface and bottom surface of each sparging element are configured to be parallel with a bottom of the benchtop bioreactor.

In some embodiments, the tubing is in gaseous communication with the sparging elements and a gas supply source.

In some embodiments, the tubing is flexible or bendable hollow tube.

In some embodiments, the tubing is formed of glass or polymer materials.

In some embodiments, the tubing comprises a first portion which is substantially vertical and a second portion which is substantially horizontal. In some embodiments, the second portion connects to the base.

In some embodiments, the base further comprises a connector tube. In some embodiments, the connector tube connects two sparging elements to the second portion, the connector tube intersecting the second portion.

In some embodiments, the second portion connects to a sparging element.

In some embodiments, the tubing further comprises a third portion which is substantially vertical. In some embodiments, the first portion is connected to a first end of the second portion, and an opposite end of the second portion is connected to the third portion. In some embodiments, each sparging element is interchangeable.

In some embodiments, the sparging elements are formed of a non-leachable, non-extractable material.

In some embodiments, the benchtop bioreactor is a 500 ml capacity benchtop bioreactor. In some embodiments, a height of each sparging element is in a range of about 3 mm to about 5 mm. In some embodiments, the height is about 4 mm. In some embodiments, a width of each sparging element is in a range of about 10 mm to about 20 mm. In some embodiments, the width is about 15 mm. In some embodiments, a length of each sparging element is in a range of about 20 mm to about 30 mm. In some embodiments, the length is about 24 mm.

In some embodiments, the benchtop bioreactor is a perfusion bioreactor. In some embodiments, the benchtop bioreactor is a perfusion spinner flask.

In some embodiments, the tubing of the macrosparger extends through an opening or port of the benchtop bioreactor. In some embodiments, the opening or port is disposed on a lid of the benchtop bioreactor. In some embodiments, the lid is removably attached to the benchtop bioreactor.

In some embodiments, the tubing is configured to follow an interior wall of the bioreactor. In some embodiments, the base of the macrosparger rests on the bottom of the bioreactor.

In some embodiments, the base of the macrosparger rests in a space below a bottom end of a mixer disposed in the bioreactor. In some embodiments, the mixer comprises an impeller attached to the bottom end. In some embodiments, the mixer is rotatable by a magnetic stir plate located external to the bioreactor.

Additional aspects of the present disclosure will be set forth, in part, in the following detailed description, figures, and claims, and in part will be derived from the detailed description, or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

FIG. 1 shows a perspective view of a macrosparger according to an embodiment of the present disclosure.

FIG. 2 shows a top view of a macrosparger according to an embodiment of the present disclosure.

FIG. 3 shows a perspective view of a macrosparger in a benchtop bioreactor according to an embodiment of the present disclosure.

FIG. 4 shows a perspective view of a macrosparger in a benchtop bioreactor according to an embodiment of the present disclosure.

FIG. 5 shows a top view of a macrosparger in a benchtop bioreactor according to an embodiment of the present disclosure.

FIG. 6 shows a top view of a macrosparger according to an embodiment of the present disclosure.

FIG. 7 shows a perspective view of a macrosparger according to an embodiment of the present disclosure.

FIG. 8 shows a perspective view of a macrosparger according to an embodiment of the present disclosure.

FIG. 9 shows a perspective view of a macrosparger according to an embodiment of the present disclosure.

FIG. 10 shows a perspective view of a macrosparger according to an embodiment of the present disclosure.

FIG. 11 shows a perspective view of a macrosparger according to an embodiment of the present disclosure.

FIG. 12 shows a side view of a macrosparger in a benchtop bioreactor according to an embodiment of the present disclosure.

FIG. 13 shows an illustration of a macrosparger in a benchtop bioreactor system according to an embodiment of the present disclosure.

FIG. 14 shows graphical images depicting the effect of the gas flow rate on the mass transfer rate within a 500 mL spinner flask.

### DETAILED DESCRIPTION

In an aspect, the subject matter described herein is directed to a macrosparger, or macrosparger, for use in benchtop bioreactors. Macrospargers according to embodiments described herein allow for a higher mass transfer rate with less shear damage and foam formation compared to commercially available spargers conventionally used in benchtop bioreactors. The macrosparger embodiments described herein are configured to provide a high level of dissolved oxygen (greater than about 45 l/hr) at high cell densities, such as cell densities that are greater than 100×10\hat{ }6 cells/mL. The macrosparger comprises a plurality of sparging elements for aeration, and the gas flow rate, number of holes, and size of holes for each sparging element is independently controllable.

In some embodiments, the macrosparger is configured to provide a high mass transfer rate within the benchtop bioreactor with less formation of foam. For example, a high mass transfer rate may be a mass transfer rate of greater than 45 l/hr. In some embodiments, the mass transfer rate may be about 50 l/hr. In some embodiments, different sparging elements are configured to achieve different desired mass transfer rates, wherein the differences are achieved by providing a different number of the plurality of openings and/or different size of the plurality of openings on the top surface of the sparging element.

The macrosparger described in embodiments herein allows for transfer of gas into cell culture media in a gentle and efficient manner. Compared to conventional spargers used in benchtop bioreactors, the macrosparger described herein provides an improved carbon dioxide (CO₂) removal rate within the bioreactor and allows for an increase in mass transfer rate and an increase in the gas-liquid contact area. The macrosparger described herein allows for a more uniform distribution of bubbles throughout the bioreactor, control of the gas entrance velocity and shear at the surface of the sparger, and a design that is easy to scale up.

The design is easy to scale up because of the geometry of the macrosparger. For example, during scaleup, it is important to keep the mass transfer rate constant across different scales which is achievable using the macrosparger configuration of embodiments described herein by changing the hole counts in the sparging elements at different scales. Also, using the macrosparger configuration of embodiments described herein allows for the gas entrance velocity to be kept constant across different scales.

The sparger includes a plurality of substantially oval-shaped sparging elements which can accommodate different sized diameters of holes or openings, as well as different numbers of holes or openings on a top surface of each sparging element. In some embodiments, the sparging elements are interchangeable and removable. The quantity of the sparging elements depends on the desired mass transfer rate. Each substantially oval-shaped sparging element can be connected to a source of gas which is independently controllable. Thus, the desired mass transfer rate and gas entrance velocity can be achieved by adjusting the flow of gas to each substantially oval-shaped sparging element.

Aspects of embodiments described herein are directed to a macrosparger for aerated benchtop bioreactors to supply oxygen for seed train intensification processes. Using embodiments of the macrosparger described herein, a high mass transfer rate can be achieved with less formation of foam, which is a main challenge in case of microspargers that are conventionally used with benchtop bioreactors.

In an aspect according to embodiments described herein, a macrosparger is provided that comprises a plurality of substantially oval-shaped sparging element. Each sparging element can accommodate a plurality of openings or holes on a top surface of the sparging element. Different sparging elements may have different numbers of holes or openings on the top surface of each sparging element. Furthermore, each hole or opening on one sparging element may have a different size or diameter than each hole or opening on another sparging element. The different sizes of the openings and number of the openings on the surface of each sparging element results in a high mass transfer rate.

The macrosparger may be formed of any suitable material. In some embodiments, the components of the macrosparger are formed of different materials. In some embodiments, the tubing of the macrosparger is formed of a first material and the sparging elements are formed of a second material.

The tubing may be formed of any material suitable for cell culture use. In some embodiments, the tubing is formed from a polymer or glass material. Nonlimiting examples of polymer material tubing include silicone tubing and polyethylene or high-density polyethylene tubing. In some embodiments, the glass material may comprise a borosilicate glass or proprietary glass formulation, such as Gorilla® glass (Corning Incorporated, Corning, NY).

The plurality of sparging elements may be constructed of any suitable material. In some embodiments, the sparging elements are formed of a non-leachable, non-extractable material. In some embodiments, the material is a liquid impermeable, gas impermeable material. Nonlimiting examples of materials that are not leachable or extractable include stainless steel (SS), polyvinylidene difluoride (PVDF), polyethylene (PE), or other polymer materials that are not leachable and extractable.

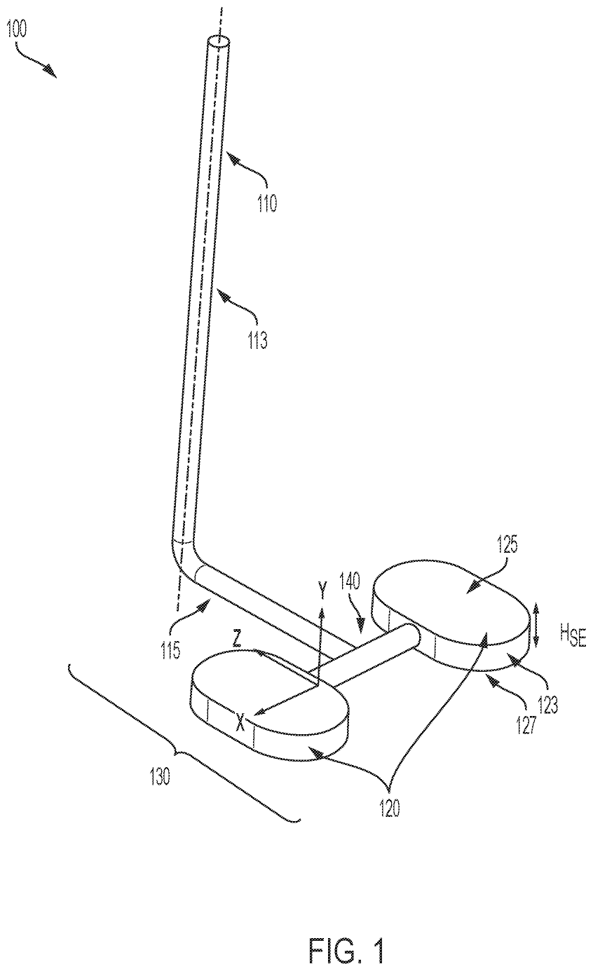

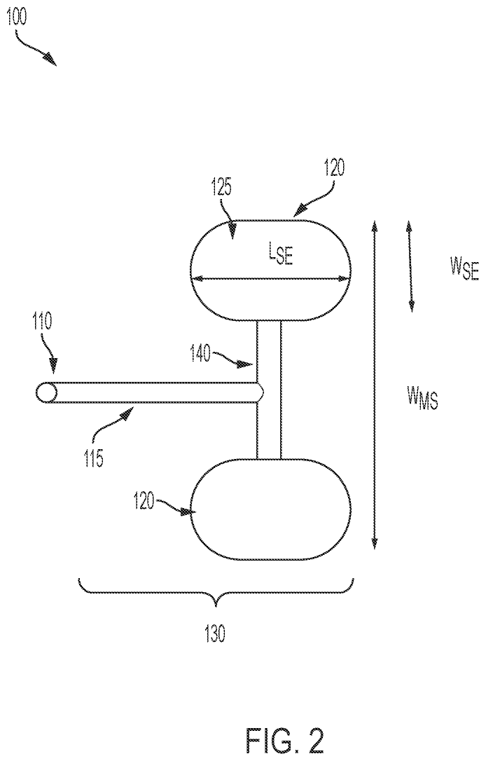

FIG. 1 shows a perspective view of an embodiment of a macrosparger 100, and FIG. 2 shows a top view of the macrosparger 100. The macrosparger comprises a tubing 110 and a base 130. The tubing 110 comprises a hollow gas impermeable tube that may be bendable or flexible. The tubing 110 comprises a substantially vertical first portion 113. The tubing 110 further comprises a second portion 115 that extends substantially perpendicular from the first portion 113. The second portion 115 extends in a substantially horizontal direction towards the base 130 of the macrosparger. The second portion 115 is connected to the base 130 at a center of a connector tubing 140.

A width of the sparger footprint or the base of the macrosparger is designated by W_{MS} and may be any suitable width that is smaller than a diameter of the bioreactor to be used. The connector tubing 140 is a hollow tubing in gaseous communication with the tubing 110 and the plurality of sparging elements 120. As shown in FIG. 1 and FIG. 2, the plurality of sparging elements comprise a first sparging element and a second sparging element. Each sparging element comprises a substantially oval-shaped top surface 125 connected to a substantially oval-shaped bottom surface 127 by a sidewall 123 to form a compartment in the sparging element. The length of the sparging element is designated as LSE, and the width of the sparging element is designated as W_{SE}. To form the substantially oval-shaped top surface, the length should be longer than the width. The height of each sparging element, H_{SE}, may be any suitable height that allows for desired aeration in a bioreactor in a space at a bottom of a bioreactor. Such a space may be restricted by the bottom of the bioreactor and a mixer that may extend toward the bottom of the bioreactor. As a nonlimiting example, H_{SE} may be in a range of about 3 mm to about 5 mm when the available bioreactor space for the macrosparger base is about 6 mm. The compartment or interior of each sparging element 120 is configured to be in gaseous communication with an interior of a bioreactor through a plurality of openings or holes on the top surface 125 of each sparging element 120.

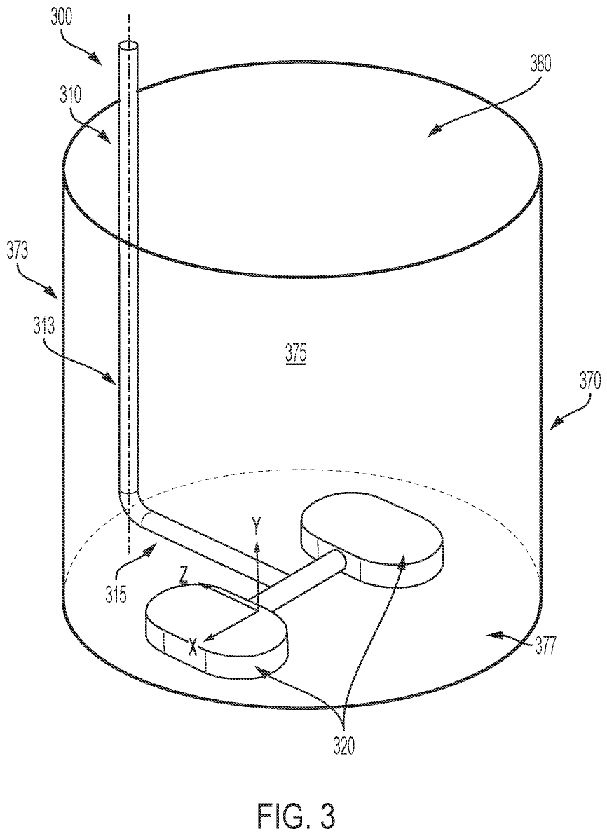

In FIG. 3, the macrosparger 300 is positioned in bioreactor 370. The tubing 310 extends through an opening or port 380 of the bioreactor 370. The first portion 313 of the tubing 310 extends substantially vertically along the sidewall 373 of the bioreactor 370, and the tubing 310 bends so that the second portion 315 of the tubing 310 (and base 330 including the plurality of sparging elements 320 and connector tube 340) extends substantially horizontally along a bottom 377 of the bioreactor 370.

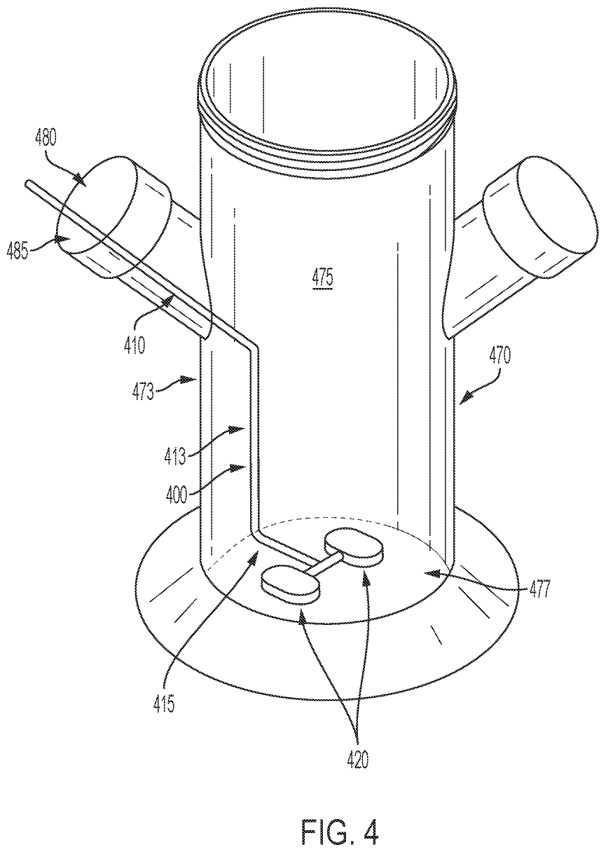

In FIG. 4, macrosparger 400 is positioned in bioreactor 470. The tubing 410 extends through a lid or cap 485 at an opening or port 480 of the bioreactor 470. The tubing 410 bends so that the first portion 413 of the tubing 410 extends substantially vertically along the sidewall 473 of the bioreactor 470, and the tubing 410 bends again so that the second portion 415 of the tubing 410 (and base 430 including the plurality of sparging elements 420 and connector tube 440) extends substantially horizontally along a bottom 477 of the bioreactor 470.

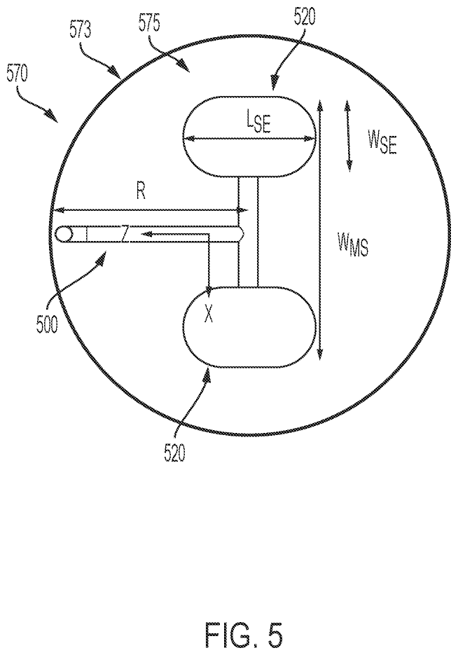

FIG. 5 shows a top view of macrosparger 500 positioned in bioreactor 570. The bioreactor 570 comprises a sidewall 573 that forms a cylindrical vessel having radius R and an interior volume 575. For purposes of FIG. 5, the bioreactor is a 500 mL spinner flask having radius R of about 37 mm. In this embodiment, the length of the sparging element, LSE, may be in a range from 20 mm to 30 mm, and in some embodiments may be about 24 mm. In this embodiment, the width of the sparging element, W_{SE}, may be in a range from 10 mm to 20 mm, and in some embodiments may be about 15 mm. In this embodiment, the width of the base of the macrosparger or macrosparger footprint, W_{MS}, may be about 50 mm.

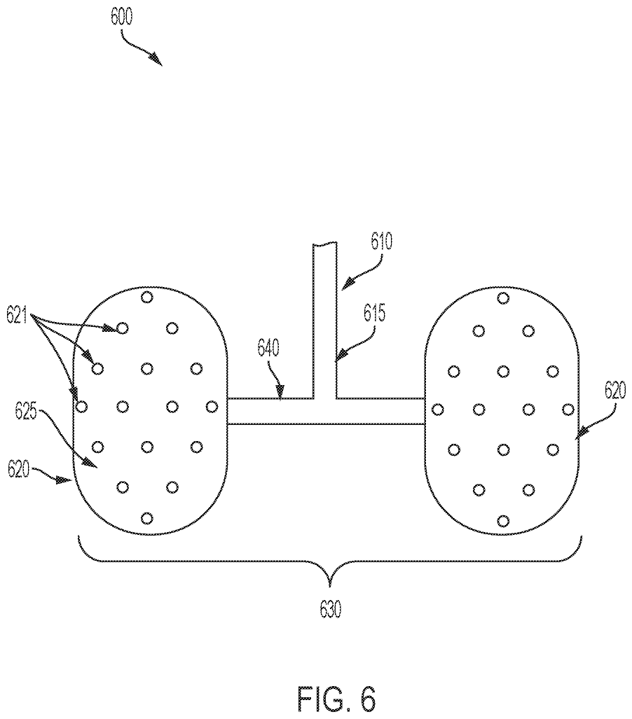

FIG. 6 shows a close-up view of an embodiment of a base 630 of a macrosparger 600 wherein the plurality of openings or holes 621 are visible. A connector tubing 640 is disposed from a side of a first sparging element 620 to a side of a second sparging element 620. The connector tubing 640 intersects the second portion 615 of the macrosparger tubing 610. The first and second sparging elements 620 are arranged in the same direction or orientation at either side of the connector tubing 640. Each sparging element 620 comprises a substantially oval-shaped top surface 625. The top surface 625 comprises a plurality of openings 621. In the embodiment shown in FIG. 6, the plurality of openings comprises 16 openings or holes uniformly spaced on the top surface 625. The openings may be circular and may have any suitable diameter. For example, the diameter of each opening may be about 100 microns. In some embodiments, such as wherein a lower mass transfer rate is desired, the diameter of the hole or opening may be larger than 100 microns. In addition, the number of holes or openings can vary based on the need of the application. For example, in some embodiments, the number of holes in the plurality of holes or openings is greater than 16. In some embodiments, the number of holes in the plurality of holes or openings is fewer than 16.

In some embodiments, each sparging element in the plurality of sparging elements may be arranged or positioned in a same orientation or direction, for example, longer sides of one substantially oval-shaped sparging element may be arranged parallel to longer sides of another substantially oval-shaped sparging element. In some embodiments, one or more sparging elements in the plurality of sparging elements may be arranged or positioned in a perpendicular orientation or direction from other sparging elements in the plurality of sparging elements. For example, longer sides of one substantially oval-shaped sparging element may be arranged perpendicular to longer sides of another substantially oval-shaped sparging element.

In some embodiments, the substantially oval-shaped sparging elements may be in gaseous communication with the connector tube or second portion of the macrosparger tubing by connecting to connector or second portion at a side of the sparging elements. In some embodiments, the side of the sparging element may comprise a longer side of the substantially oval-shaped sparging element. In some embodiments, the side of the sparging element may comprise a shorter side of the substantially oval-shaped sparging element.

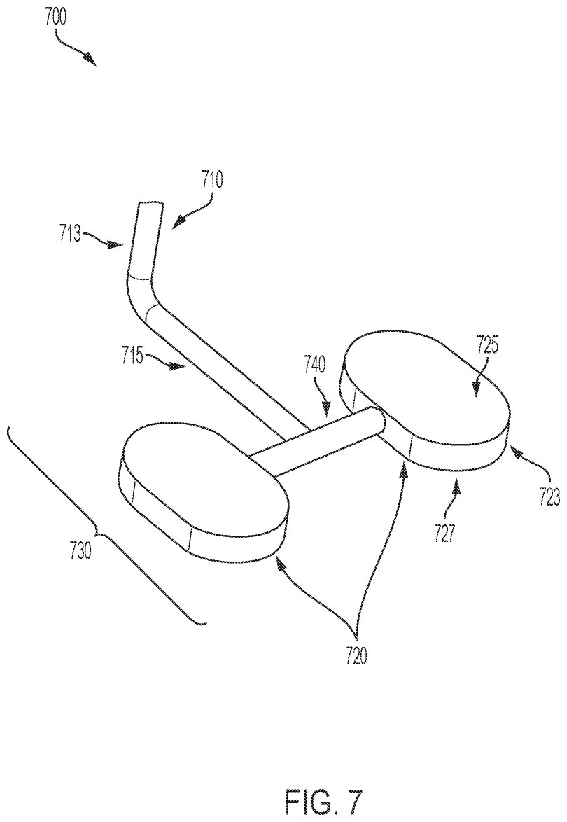

In some embodiments, the substantially oval-shaped sparging elements on a base of the macrosparger are interchangeable and removable. The number or quantity of the aeration elements (substantially oval-shaped sparging elements) depends on the desired mass transfer rate. Different configurations of the plurality of sparging elements for embodiments of the macrosparger are shown in FIGS. 7-11.

FIG. 7 shows an embodiment of a macrosparger 700. A connector tubing 740 is disposed from a side of a first sparging element 720 to a side of a second sparging element 720. The second portion 715 of the macrosparger tubing 710 extends substantially perpendicular from the first portion 813. The second portion 715 connects to the connector tubing 740 perpendicular to the connector tubing and at a center of the connector tubing. The first and second sparging elements 720 are arranged in the same direction or orientation at either side of the connector tubing 740. Each sparging element 720 comprises a substantially oval-shaped top surface 725 and a substantially oval-shaped bottom surface 727, with the top surface and bottom surface connected by a sidewall 723 to form a compartment within the sparging element. The top surface 725 comprises a plurality of openings (not shown), and the compartment within the sparging element is in gaseous communication with an interior of a bioreactor through the plurality of openings.

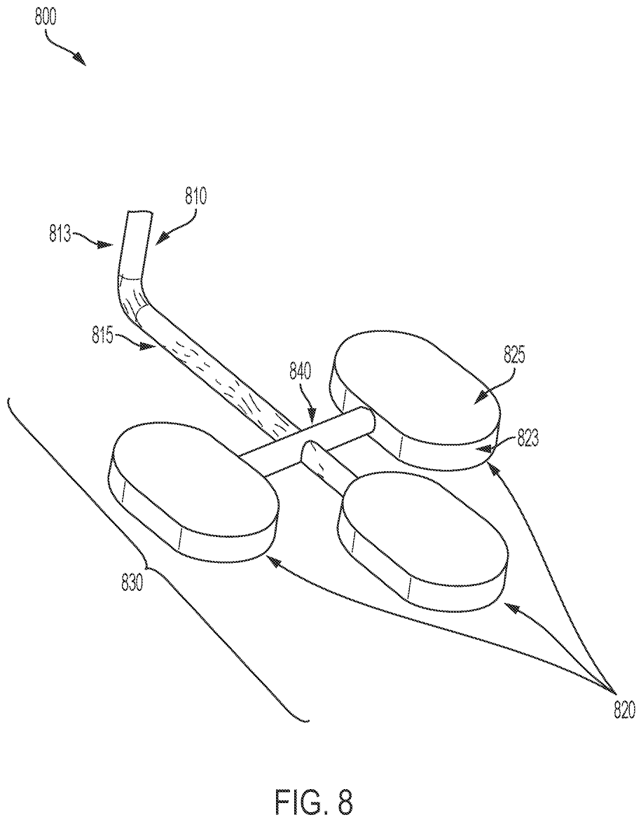

FIG. 8 shows an embodiment of a macrosparger. In the embodiment shown in FIG. 8, the plurality of sparging elements comprises three sparging elements 820. A connector tubing 840 is disposed at side of a first sparging element 820 and extends to a side of a second sparging element 820. The connector tubing 840 intersects the second portion 815 of the macrosparger tubing 810. The second portion 815 of the macrosparger tubing 810 extends horizontally from the first portion 813 of the macrosparger tubing 810 to a side of a third sparging element 820. In the embodiment shown, the first, second, and third sparging element have the same direction or orientation.

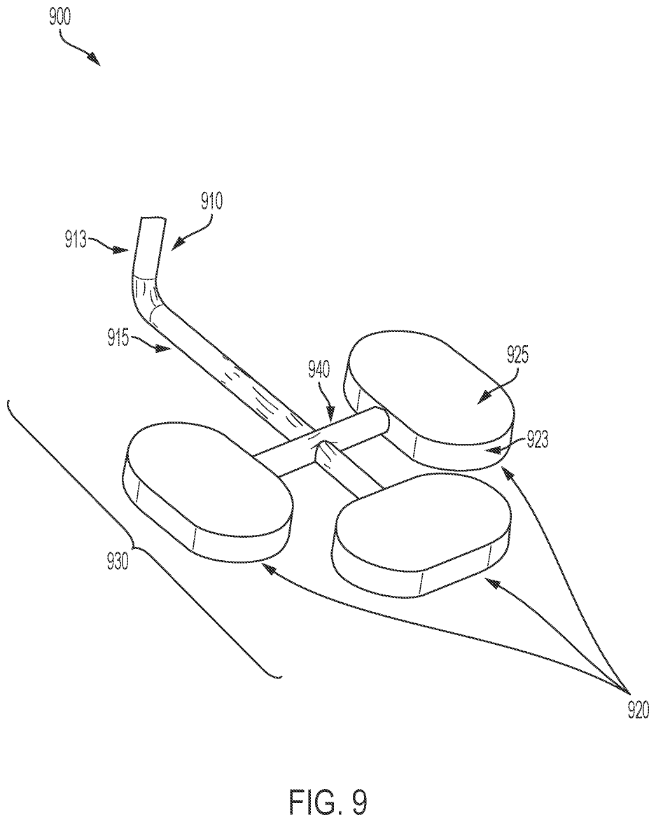

FIG. 9 shows an embodiment of a macrosparger. In the embodiment shown in FIG. 9, the plurality of sparging elements comprises three sparging elements 920. A connector tubing 940 is disposed at side of a first sparging element 920 and extends to a side of a second sparging element 920. The connector tubing 940 intersects the second portion 915 of the macrosparger tubing 910. The second portion 915 of the macrosparger tubing 910 extends horizontally from the first portion 913 of the macrosparger tubing 910 to a side of a third sparging element 920. In the embodiment shown, the first and second sparging elements share a same direction or orientation, while the third sparging element is disposed so that the direction or orientation is perpendicular to that of the first and second sparging elements.

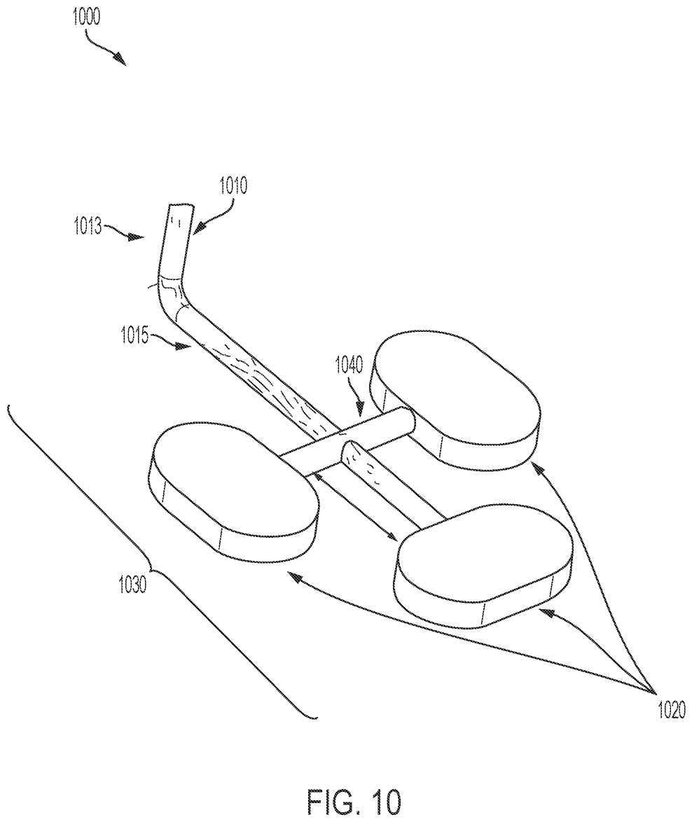

FIG. 10 shows an embodiment of a macrosparger directed to achieving improved CO₂ removal rate from the bioreactor. In the case of CO₂ accumulation, the macrosparger comprises three substantially oval-shaped sparging elements. A connector tubing 1040 is disposed at side of a first sparging element 1020 and extends to a side of a second sparging element 1020. The connector tubing 1040 intersects the second portion 1015 of the macrosparger tubing 1010. The second portion 1015 of the macrosparger tubing 1010 extends horizontally from the first portion 1013 of the macrosparger tubing 1010 to a side of a third sparging element 1020. In the embodiment shown, the first and second sparging elements share a same direction or orientation, while the third sparging element is disposed so that the direction or orientation is perpendicular to that of the first and second sparging elements. The third sparging element may be positioned further away from a center of the bioreactor, and thus further away from the impeller, in order to improve CO₂ removal rate from the bioreactor.

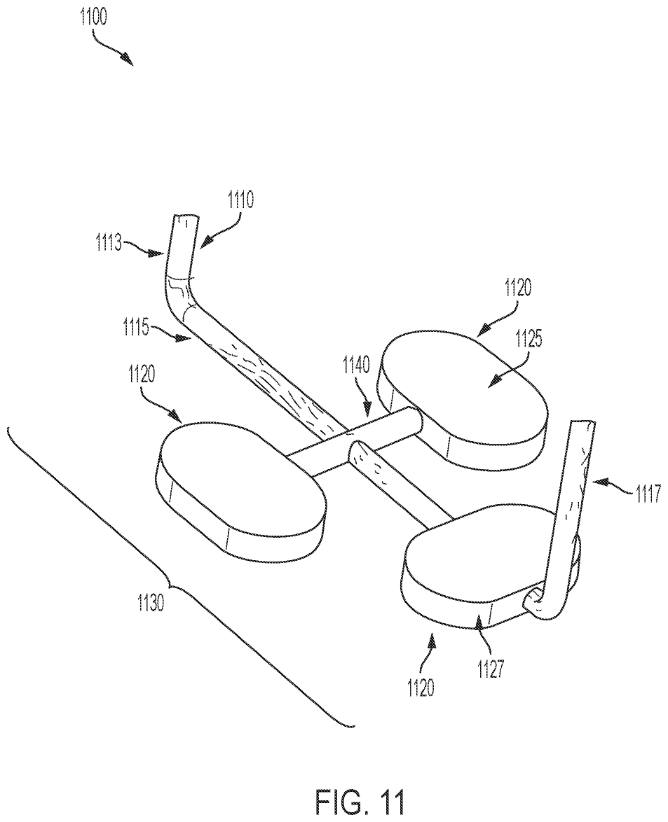

FIG. 11 shows an embodiment of a macrosparger directed to having more than one gas connection. For example, each substantially oval-shaped sparging element can be connected to a source of gas which is independently controllable. Thus, the desired mass transfer rate and gas entrance velocity can be achieved by adjusting the flow of gas to each substantially oval-shaped sparging element.

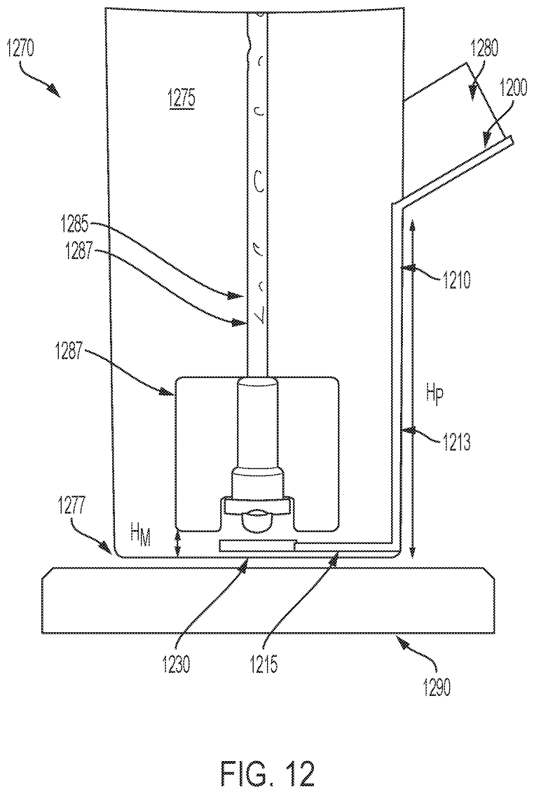

FIG. 12 shows an embodiment of a macrosparger 1200 positioned in a bioreactor 1270. The bioreactor 1270 shown is a perfusion spinner flask and is positioned on a magnetic stir plate 1290. The bioreactor 1270 comprises a vessel having a bottom 1277 and an interior volume 1275. The bioreactor comprises one or more openings or ports 1280 towards a top of the bioreactor. The macrosparger 1200 extends through one such opening or port 1280. The macrosparger tubing 1210 extends through the opening or port 1280, a first portion 1213 may bend to travel substantially vertically down a sidewall of the bioreactor, wherein H_{P} is the height from the bottom of the bioreactor to the port, and a second portion 1215 may bend again to travel substantially horizontally along the bottom of the bioreactor to the base 1230 of the macrosparger. The base 1230 of the macrosparger may be positioned at a center of the bottom 1277 of the bioreactor. The bioreactor 1270 may comprise a mixer 1285 having a shaft 1287 and an impeller 1289 located at an end or bottom of the shaft 1287. The base 1230 of the macrosparger may be positioned below the bottom of the mixer 1285 and above the bottom 1277 of the bioreactor in a space having a height H_{M}. In some embodiments, the bioreactor is a 500 mL spinner flask, H_{P} is about 91 mm, and H_{M} is about 6 mm.

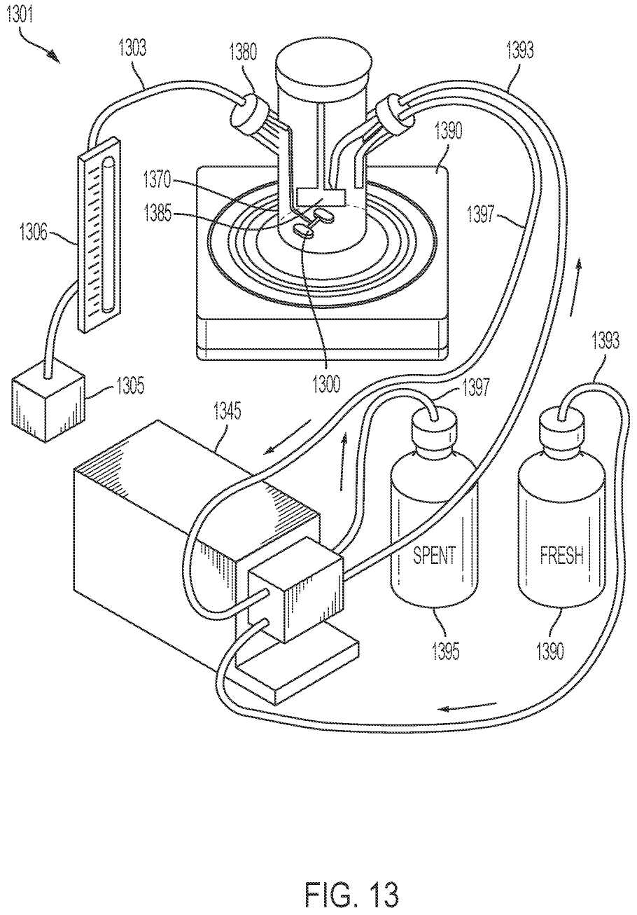

Referring to FIG. 13, there is a schematic illustrating the basic components of a system 1301 comprising a macrosparger 1300 in a benchtop bioreactor 1370 in accordance with an embodiment of the present disclosure. The bioreactor 1370 may include an interior volume 1375 formed by a sidewall and a bottom, an opening or port 1380, and an optional lid for the opening or port.

A fresh media bottle 1390 may have its contents, namely fresh media, pumped by a pump head of a peristaltic pump 1345 into the interior volume 1375, while the spent media and cell secreted material may be pumped by another pump head of the peristaltic pump 1345 out of the interior volume 1375 of the bioreactor 1370 into the spent media bottle 1395. in combination with the macrosparger 1300, an air pump 1305, also called a sparging pump, and an airflow meter 1306 may be used to control the amount of aeration that the cells experience within the interior volume 1375 of the bioreactor 1370. The magnetic stir plate 1390 may use a rotating magnet container therein to rotate an impeller and shaft of a mixer within the interior volume 1375 of the bioreactor 1370. In view of the foregoing, there is disclosed a benchtop bioreactor 1370 which has an interior volume 1375 (inner compartment) where cells can be cultivated in a growth medium through agitation provided by a mixer 1385. Fresh media 1390 may be continuously fed to the interior volume 1375 (inner compartment) of the bioreactor 1370 through a feed tube 1393, while nutrient-depleted media 1395 may flow out of the interior volume 1375 of the bioreactor 1370 through a vacuum port (e.g., spent media tube 1397).

In embodiments, a lid may be removably attached to an opening, aperture, or port of the bioreactor, or may be permanently attached to the bioreactor. The lid may be attachable (e.g., screwed, pushed-on) to the bioreactor in order to cover the opening. In embodiments, then lid is integral to the bioreactor, allowing the bioreactor, once assembled, to be a closed, integral device. In embodiments, the lid may be removable, which allows the bioreactor to be disassembled by the user and the contents to be accessed by the user.

In some embodiments, a fresh media port and a spent media port may both extend through a lid. In some embodiments, a fresh media port and a spent media port may extend through an opening or aperture of the bioreactor. The fresh media port is configured to receive a fresh media tube that has an end located in the interior volume of the bioreactor. The fresh media tube is used to supply fresh media to the interior volume of the bioreactor. The spent media port is configured to receive a spent media tube that has an end located in the interior volume of the bioreactor. The spent media tube is used to remove the spent media and the cell secreted material (e.g., recombinant protein, antibody, virus particles, DNA, RNA, sugars, lipids, biodiesel, inorganic particles, butanol, metabolomic byproducts) from the bioreactor.

In embodiments, any suitable mixer may be used. The mixer may comprise a shaft with an impeller at the bottom or end of the shaft. The impeller and the shaft may both be disposed within the interior volume of the bioreactor. For example, the impeller may be attached to a bottom end of the shaft while another end of the shaft may be rotatably attached to and extending down ward from a removable lid. The mixer may be magnetic. The impeller may be rotated by a magnetic stir plate located under the bioreactor.

In some embodiments, the benchtop bioreactor system may further comprise a magnetic stir plate located external to the bioreactor. The magnetic stir plate is configured to rotate the impeller and the shaft.

The gas sparger according to embodiments of the disclosure, also referred to as a macrosparger, may optimize the availability of oxygen to cells contained in the benchtop bioreactor. The macrosparger may be used to add oxygen to the media in the interior volume of the bioreactor.

In some embodiments, benchtop bioreactor systems further comprise a plurality of sensors (e.g., temperature, DO₂, CO₂, pH, cell density). Optionally, a sensor port may be connected to a sensor that has an end located in the interior volume of the bioreactor. For example, the sensor can be a dissolved oxygen (DO₂) sensor, a carbon dioxide (CO₂) sensor, a pH sensor, a cell density sensor, a glucose sensor, or a flow or shear stress and temperature sensor, or any other sensor.

The bioreactor can be operated continuously. The assembled macrosparger, benchtop bioreactor, and other system components may be gamma irradiated, e-beam sterilized, ultra-violet (UV) sterilized, ethanol sterilized, or gas sterilized. In some embodiments, the macrosparger and benchtop bioreactor system may be positioned inside an incubator for use during incubation.

The bioreactor can be plastic, glass, ceramic or stainless steel. In embodiments, the bioreactor may be a rigid container or may be a flexible bag.

The bioreactor can be any size. In some embodiments, the bioreactor has a size of about 0.1 liter to about 1000 liters or more. In some embodiments, the benchtop bioreactor may be a perfusion bioreactor such as that described in US 2019/0048305, the contents of which are incorporated herein. In some embodiments, the bioreactor is a 500 mL spinner flask perfusion bioreactor.

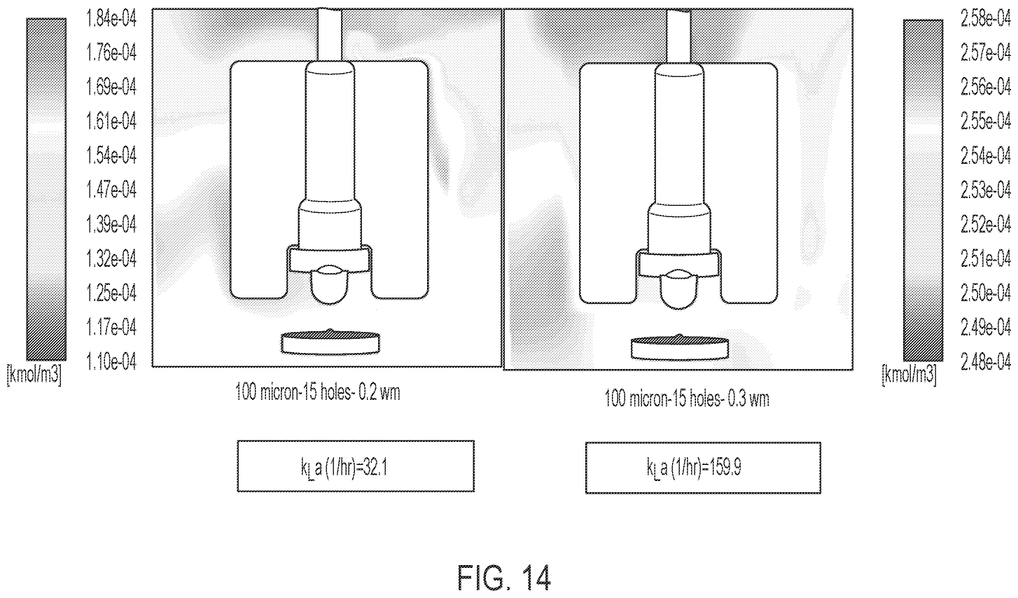

FIG. 14 shows graphical images depicting the effect of the gas flow rate on the mass transfer rate within a 500 mL spinner flask. For mammalian cell culture, the gas flow rate through a sparger depends on the type of the sparger. For macrospargers, such as macrospargers according to the embodiments described herein, the total gas flow rate is between 0.1-0.3 vvm (gas volumetric flow rate per unit volume of culture media). Therefore, in order to evaluate the effect that the gas flow rate has on the mass transfer rate within the 500 mL spinner flask, two different gas flow rates were considered. As shown in FIG. 14, the graphical image on the left considers a gas flow rate of 0.2 vvm, while the graphical image on the right considers a gas flow rate of 0.3 vvm. The graphical images on the left and the right both consider the same diameter of holes (100 micron) and the same number of holes (15 holes) for gas sparging elements. As FIG. 14 shows, with the same hole diameter and same number of holes, increasing the gas flow rate from 0.2 vvm to 0.3 vvm enhanced the mass transfer coefficient (k_{L}a) from 32.1 (l/hr) to 159.9 (l/hr), respectively. In addition, at the maximum recommended gas flow rate of 0.3 vvm, the obtained mass transfer rate of 159.9 (l/hr) was much higher than the mass transfer rate of about 50 (l/hr) required for seed train intensification. Thus, using a macrosparger according to embodiments described herein, the oxygen demands of high cell density culture can be met at lower gas flow rates, which in turn results in minimizing cell damage and foaming in the bioreactor.

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising.” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been illustrated in the accompanying Drawings and described in the foregoing Detailed Description, it should be understood that the disclosure is not limited to the disclosed embodiments, but is capable of numerous rearrangements, modifications and substitutions without departing from the disclosure as set forth and defined by the following claims.

1. A macrosparger for a benchtop bioreactor comprising:
a tubing; and
a base comprising a plurality of sparging elements in gaseous communication with the tubing and an interior of a benchtop bioreactor, each sparging element comprising:
a cylindrical body having an internal compartment formed by a top surface and a bottom surface, the top surface and bottom surface connected by a sidewall,
wherein the top surface and bottom surface are substantially oval-shaped.

2. The macrosparger of claim 1, wherein the top surface and bottom surface are substantially flat.

3. The macrosparger of claim 1, wherein the top surface comprises a plurality of openings, the compartment in gaseous communication with the interior of the benchtop bioreactor through the plurality of openings.

4. The macrosparger of claim 1, wherein each of the openings in the plurality of openings is uniformly spaced on the top surface.

5. The macrosparger of claim 1, wherein the top surface and bottom surface of each sparging element are configured to be parallel with a bottom of the benchtop bioreactor.

6. The macrosparger of claim 1, wherein the tubing is in gaseous communication with the sparging elements and a gas supply source.

7-8. (canceled)

9. The macrosparger of claim 1, wherein the tubing comprises a first portion which is substantially vertical and a second portion which is substantially horizontal,
wherein the second portion connects to the base, wherein the base further comprises a connector tube,
wherein the connector tube connects two sparging elements to the second portion, the connector tube intersecting the second portion, and
wherein the second portion connects to a sparging element.

10-13. (canceled)

14. The macrosparger of claim 9, wherein the tubing further comprises a third portion which is substantially vertical, wherein the first portion is connected to a first end of the second portion, and an opposite end of the second portion is connected to the third portion.

15. (canceled)

16. The macrosparger of claim 1, wherein each sparging element is interchangeable.

17. The macrosparger of claim 1, wherein the sparging elements are formed of a non-leachable, non-extractable material.

18. The macrosparger of claim 1, wherein the benchtop bioreactor is a 500 mL capacity benchtop bioreactor.

19. The macrosparger of claim 18, wherein a height of each sparging element is in a range of about 3 mm to about 5 mm.

20. (canceled)

21. The macrosparger of claim 18, wherein a width of each sparging element is in a range of about 10 mm to about 20 mm.

22. (canceled)

23. The macrosparger of claim 18, wherein a length of each sparging element is in a range of about 20 mm to about 30 mm.

24. (canceled)

25. The macrosparger of claim 1, wherein the benchtop bioreactor is a perfusion bioreactor.

26. The macrosparger of claim 1, wherein the benchtop bioreactor is a perfusion spinner flask,
wherein the base of the macrosparger rests on the bottom of the bioreactor, and
wherein the base of the macrosparger rests in a space below a bottom end of a mixer disposed in the bioreactor.

27. The macrosparger of claim 1, wherein the tubing of the macrosparger extends through an opening or port of the benchtop bioreactor, and wherein the opening or port is disposed on a lid of the benchtop bioreactor.

28. (canceled)

29. The macrosparger of claim 27, wherein the lid is removably attached to the benchtop bioreactor.

30. The macrosparger of claim 1, wherein the tubing is configured to follow an interior wall of the bioreactor.

31-33. (canceled)

34. The macrosparger of claim 26, wherein the mixer is rotatable by a magnetic stir plate located external to the bioreactor.
